(19) United States
(12) Patent Application Publication
(10) Pub. No.: US 2008/0139638 A1
(43) Pub. Date: Jun. 12, 2008

(54) METHOD OF INCREASING GROWTH HORMONE SECRETION

(76) Inventors: MARVIN HEUER, MISSISSAUGA (CA); BRAD PILON, MISSISSAUGA (CA)

Correspondence Address: TORYS LLP, 79 WELLINGTON ST. WEST, SUITE 3000, TORONTO, ON, M5K 1N2, omitted

(21) Appl. No.: 11/567,465

(22) Filed: Dec. 6, 2006

(51) Int. Cl. A61K 31/4045

(52) U.S. Cl. 514/419

(57) ABSTRACT

The present invention relates to a method of administering to a mammal between from about 0.01 mg and about 4.9 mg of melatonin followed by physical exercise wherein said exercise occurs within 60 minutes following said administration of melatonin to increase serum growth hormone levels.

### FIELD OF THE INVENTION

The present invention relates to a method for increasing growth hormone secretion in an individual.

### BACKGROUND

Growth hormone, also known as somatotropin, is a hormone which is secreted by the adenohypohysis of the pituitary gland. It has long been implicated to play a role in cellular proliferation and differentiation, metabolic regulation and protein synthesis. Growth hormone binding protein (GHBP) binds nearly 50% of the growth hormone in circulation, which in its free, unbound state is able to bind to the extracellular domain of the growth hormone receptor. Furthermore, the anabolic role of growth hormone and its relative free-circulating levels are able to influence the activity of various growth factors such as insulin-like growth factor-1 (IGF-1). In addition, the majority of growth hormone secretion is controlled by hormonal signals from the hypothalamus, which itself receives signals from virtually all areas of the nervous system. As such, growth hormone secretion appears to be related to, and conceivably regulated by the hypothalamus-pituitary-adrenal (HPA) axis.

Melatonin, also known as Melatonine, Circadin, N-Acetyl-5-methoxytryptamine, and 5-Methoxy-N-acetyltryptamine, is naturally produced in the brain by the pineal gland and has been shown to stimulation the production of growth hormone as well as reduce free-radical damage. Evidence suggests that melatonin plays a role in modulating pituitary gland secretions such as growth hormone. Furthermore, melatonin follows a circadian rhythm and is thus principally controlled by a shift from light to dark within the environment. Growth hormone secretion is related to sleep, and during the forth stage of sleep, an increase is observed. Small doses of melatonin, less than 2 mg, administered orally have not be shown to affect the 24-hr profile of growth hormone concentration (Wright J, Aldhous M, Franey C, English J, Arendt J. The effects of exogenous melatonin on endocrine function in man. Clin Endocrinol (Oxf). April 1986;24(4):375-82.), however, doses of 1 g and 0.4 mg/kg respectively administered orally and intravenously, have been shown to have an effect on basal growth hormone levels (Valcavi R, Zini M, Maestroni G J, Conti A, Portioli I. Melatonin stimulates growth hormone secretion through pathways other than the growth hormone-releasing hormone. Clin Endocrinol (Oxf). August 1993;39(2):193-9; Smythe G A, Lazarus L. Growth hormone responses to melatonin in man. Science. Jun. 28, 1974;184(144):1373-4; Esposti D, Lissoni P, Mauri R, Rovelli F, Orsenigo L, Pescia S, Vegetti G, Esposti G, Fraschini F. The pineal gland-opioid system relation: melatonin-naloxone interactions in regulating GH and LH releases in man. J Endocrinol Invest. February 1988;11(2):103-60.), indicating a stimulatory affect of melatonin on growth hormone release.

Exogenous oral melatonin administration of both 0.5 mg and 5.0 mg has been shown to produce a significant increase in plasma growth hormone concentrations with peak values at 60 minutes being similar in amplitude. Moreover, both of the aforementioned values share similar areas under the curve as detected by two site immunoradiometric assays (Forsling M L, Wheeler M J, Williams A J. The effect of melatonin administration on pituitary hormone secretion in man. Clin Endocrinol (Oxf). November 1999;51(5):637-42.) indicating that 0.5 mg may be the maximal dose for growth hormone stimulation. This study, however, did not measure the concomitant effect of exercise with melatonin administration.

A release of growth hormone has also been shown to occur in response to single bouts of both cardiovascular and resistance exercise. At 85% of the weight of the one repetition maximum for an individual, a single bout of weight lifting exercise was shown to significantly elevate the serum level of growth hormone (Vanhelder W P, Goode R C, Radomski M W. Growth hormone responses during intermittent weight lifting exercise in men. Eur J Appl Physiol Occup Physiol. 1984;53(1):31-4.). Additionally, the serum levels of growth hormone where shown to be increased by a single set of low- and moderate-intensity (50% and 70% of one repetition maximum respectively) resistance exercise following high intensity exercise (90% of one repetition maximum) (Goto K, Sato K, Takamatsu K. A single set of low intensity resistance exercise immediately following high intensity resistance exercise stimulates growth hormone secretion in men. J Sports Med Phys Fitness. June 2003;43(2):243.).

Exogenous melatonin administered orally prior to bicycle exercise at 70% VO₂ₘₐₓ was shown to cause significant increases in growth hormone when compared to placebo through a calculation of the area under the curve. In this case, 5.0 mg was administered orally 60 minutes prior to exercise and growth hormone levels were shown to peak at 30 minutes following exercise, whereas the increase in growth hormone levels in the placebo group peaked at 15 minutes following exercise (Meeking D R, Wallace J D, Cuneo R C, Forsling M, Russell-Jones D L. Exercise-induced GH secretion is enhanced by the oral ingestion of melatonin in healthy adult male subjects. Eur J Endocrinol. July 1999;141(1):22-6).

The group of Forsling et al., 1999, although using both 5.0 mg and 0.5 mg orally administered doses of melatonin, did not employ exercise as part of the protocol. The increase in growth hormone for the experiments of Forsling et al., can only be attributed to oral melatonin administration alone. The group of Meeking et al., 1999, although employing submaximal exercise as part of their protocol, did not include a 0.5 mg administered group, only employed a 5.0 mg group to determine the effects of melatonin and exercise on growth hormone secretion. There was no lower dose testing and subsequent determination of the effect of orally administered melatonin and exercise on growth hormone secretion.

U.S. Pat. No. 6,521,591, entitled “Pharmaceutical Composition for Muscular Anabolism” discloses as pharmaceutical composition comprising the daily administration of at least 5.0 mg of components which trigger anabolism, such as growth factors, 0.12 g of components which provide building blocks for the biosynthesis of muscle such as proteins and amino acids comprising a weight ratio of leucine to branched-chain amino acids between 0.5 and 3.0 and at least 3 g of anabolic facilitators such as those which facilitate biosynthesis and prevent catabolism comprising at least 1 g of creatine or its functional equivalent. The pharmaceutical composition disclosed by the inventors may further comprise between 0.5 and 10 g of melatonin per daily dose. The inventors do not disclose or suggest the combination of the aforementioned pharmaceutical composition with physical exercise or even the sole melatonin supplementation in an individual. Furthermore, the inventors contemplate melatonin dose ranges as part of the pharmaceutical composition in the order of from 0.5 g to 10 g per day.

### SUMMARY OF THE INVENTION

The foregoing needs and other needs and objectives that will become apparent for the following description are achieved in the present invention, which comprises a method of increasing growth hormone secretion in an individual. The method of the present invention comprises the steps of administering to an individual between from about 0.01 mg to about 4.9 mg of melatonin followed by physical exercise wherein said exercise occurs within about 60 minutes following said administration of melatonin.

### BRIEF DESCRIPTION OF THE DRAWINGS

FIG. 1 is a bar graph showing the level of serum growth hormone relative to baseline growth hormone levels with and without melatonin supplementation.

FIG. 2 is a bar graph showing the average relative levels of serum growth hormone following melatonin supplementation and exercise.

FIG. 3 is a line graph showing a time course and the concentration of serum group hormone for each treatment group following exercise.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a method for increasing growth hormone secretion in an individual comprising exercise and the prior administration of from about 0.01 mg to about 4.9 mg of melatonin.

A study to determine the effects of either a placebo or melatonin supplementation prophylacatically administered at doses of either 0.5 mg or 5.0 mg prior to a single about of resistance exercise on growth hormone was undertaken. Consistent with the results of Meeking et al., 1999, submaximal exercise following the oral administration of 5.0 mg of melatonin, showed an approximately 2.35—fold increase in the studies undertaken by the inventors of the present invention as compared to placebo. Surprisingly, it was discovered that the 0.5 mg dose of orally administered melatonin prior to a single bout of resistance exercise, there was a significantly greater increase in free growth hormone as compared to either the placebo group or the 5.0 mg administered group. These results indicate that, consistent with previously reported studies, the inventors have shown that exercise and melatonin act to synergistically increase growth hormone level, however, unexpectedly, the lower orally administered dose of 0.5 mg, when combined with exercise results in the greatest increase in growth hormone secretion.

### Methods and Procedures

### Participants

Thirty, healthy males who had partaken in regular resistance training for at least one year prior to the study were selected [n=30(22.72±3.28 years, 180.94±6.24 cm in height, and 82.08±9.18 kg in weight)] served as participants in the study. All of the participants passed a mandatory medical screening and were considered either as low or moderate risk or with no contradictions to exercise as outline by the American College of Sports Medicine (ACSM). Furthermore, the participants had not consumed any nutritional supplements, with the exclusion of vitamins, during a two month period prior the experiment.

### Entry/Familiarization and Baseline Strength Testing Session

Participants whom were believed to meet the eligibility criteria of the study were asked to attend an Entry/Familiarization and baseline testing session. Participants were asked to refrain from lower-body exercise for 48 hours prior to the baseline testing session. Upon reporting to the lab for the baseline testing session, participants were asked to complete a medical history questionnaire and undergo a general physical examination to determine if the eligibility criteria were met. Participants who met the eligibility criteria were familiarized with the study protocol by way of a verbal and written explanation outlining the study. An initial strength test to asses the participants one repetition maximum on the leg press exercise (Body Master, Ruston, La.) was performed. The participants' heart rate and blood pressure were monitored throughout the testing session. Following the determination of the one repetition maximum, participants were asked to perform and practice the proposed resistance exercise session in its entirety (see Training and Supplementation section) without blood sampling to familiarize themselves with the protocol. This was to also ensure that they could complete the protocol before being formally admitted to the study. At the end of the testing session, an appointment was booked with each participant for a time one week later to begin the study.

### Training and Supplementation

Participants were matched by relative strength level (strength/body weight) and then randomly assigned to one of three different protocols. The supplement protocol consisted of a double-blind with groups receiving either 0.5 mg or 5.0 if melatonin (N-Acetyl-5-methoxytryptamine) or 1.0 mg of dextrose as a placebo. Participants were allowed a light breakfast at 8:00 am, wherein they were asked to record the consumed items, and then were required to fast until 2 pm. Seventy-five minutes prior to each exercise session, (12:30 pm), an intravenous cannula was inserted into a foreman vein. At 60 minutes prior to the exercise session, a baseline blood sample was taken and the participant orally received a supplement of placebo according top that outlined above. During the 60 minutes prior to exercise, the participants were required to rest in a supine position and blood samples were taken every 15 minutes up to the initiation of the resistance exercise. The leg press exercise consisted of 7 sets of 7 slow repetitions at 85% of the weight of the respective participant's one repetition maximum. Each set was preformed over the course of 30 seconds followed by 150 seconds of rest. The entire resistance exercise session was 21 minutes in duration.

### Dietary Records

Participants were not required to adhere to a standardized diet and were asked not to change the eating habits during the course of the study. The participants were, however, required to maintain dietary records for the 48 hours prior to the resistance exercise session. The 48-hours dietary recalls were evaluated with the “Food Processor” dietary assessment software program to determine the average daily macronutrient consumption of fat, carbohydrate, and protein in the diet prior to supplementation and resistance exercise.

### Blood Collection Procedures

Venous blood samples were obtained into 10 ml vacutainer tubes via a 20 gauge intravenous catheter inserted into a foreman vein after the subcutaneous injection of a topical anesthetic (2% Xylocaine). Following the five hour fast, blood samples were obtained at 15-minute intervals commencing 60 minutes prior to the resistance exercise session and then at 15-minute intervals for 120 minutes following the resistance exercise session. Blood samples were allowed to stand for 10 minutes at room temperature and then centrifuged at 2,400 rpm for 10 minutes. The serum was then separated and stored at (−)20° C. until analysis.

### Clinical Chemistry Analysis

Using a Dade Dimension RXL clinical chemistry analyzer and Abbott Cell-Dyn 3500 hematology analyzer, blood samples were analyzed for general clinical chemistry markers, for example, glucose, total protein, blood urea nitrogen (BUN), creatinine, BUN/creatinine ratio, AST, ALT, creatine kinase (CK), lactate dehydrogenase (LDH), gamma-glutamyl transpeptide (GGT), albumin, globulin, sodium, chloride, calcium, carbon dioxide, total bilirubin, alkaline phosphatase, triglycerides, cholesterol, high density lipoproteins (HDL) and low density lipoproteins (LDL). Whereas whole blood samples were analyzed for standard cell blood counts with percentage differentials, for example, hemoglobin, hemotocrit, red blood cell counts, mean corpuscular volume (MCV), mean corpuscular hemoglobin (MCH), mean corpuscular hemoglobin in concentration (MCHC), red cell distribution width (RDW) and white blood cell counts.

### Serum Hormone Analyses

Using enzyme-linked immunosorbent assays (ELISA), and enzyme-immunosorbent assays (EIA), serum samples were assayed for growth hormone (Diagnostics Systems Laboratories, Webster, Tex.) using a Wallac Victor-1420 microplate reader from Perkin-Elmer Life Sciences. Boston, Mass. at either 450 or 405 nm respectively. An ultra-sensitive assay with a lower detection limit of 0.66 pg/ml was used to determine the levels of growth hormone without any detectable interference of circulation glycoproteins, GH-dependent peptide hormones, or growth hormone binding protein.

### Assessment of Hemodynamic Safety Markers (Heart Rate and Blood Pressure)

At each blood sampling period, the heart rate and pressure of the participants was assessed. The heart rate was determined using a Polar heart rate monitor (Polar, San Ramon, Calif.), and the blood pressure was assessed in the supine position using a mercurial sphygmomanometer employing standard procedures.

### Reported Side Effects from Supplements

At the end of the testing session, participant were asked to report in a questionnaire whether they tolerated the supplement, supplementation protocol, as well as any medical problems or symptoms with which they may have encountered throughout the study.

### Statistical Analyses

Statistical analyses were performed using factorial analysis of variance (ANOVA) with repeated measures for each criterion value. Pairwise comparisons were used to differentiate significant differences among the three groups. All statistical procedures were performed using SPSS 11.0 software and a probability level of <0.05 was adopted throughout.

### Results

### Resistance Exercise Session

The exercise bout was completed by all participants at 84.7%±0.0004 of their respective one repetition maximum. Previously, it has been shown that exercise at this level would produce a growth hormone response (Vanhelder W P, Goode R C, Radomski M W. Growth hormone responses during intermittent weight lifting exercise in men. Eur J Appl Physiol Occup Physiol. 1984;53(1):31-4.). As such, growth hormone responses were compared both pre- and post-exercise across all groups in order to differentiate and synergistic effect between the supplement and exercise.

### Dietary Analysis

From the dietary records, it was shown that their was no significant differences in the total calories or macronutrient intake of carbohydrates, fat and protein between the three studied groups during the period of 48 hours prior to the testing session (p>0.05).

### Serum Hormones

Short half-life hormones which are highly sensitive to diurnal variations, such as growth hormone, display great variability in the baseline values as was observed. As such, peak serum hormone responses before and after resistance exercise were subtracted from the baseline values of each participant in order to determine the increase in the growth hormone resulting from the treatments.

### Results and Discussion

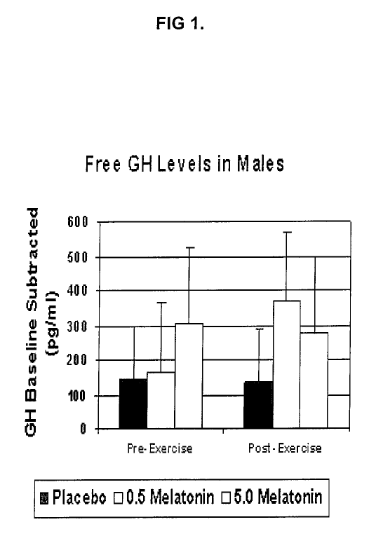

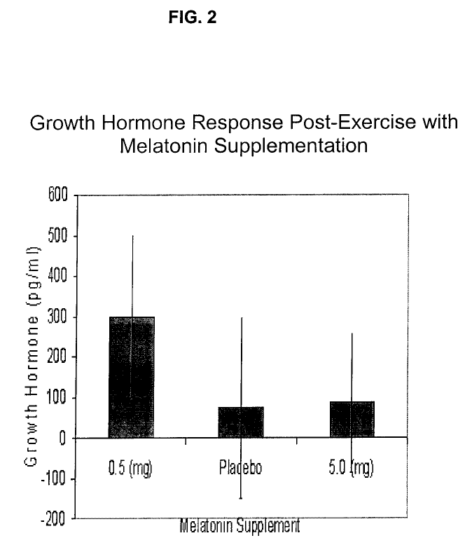

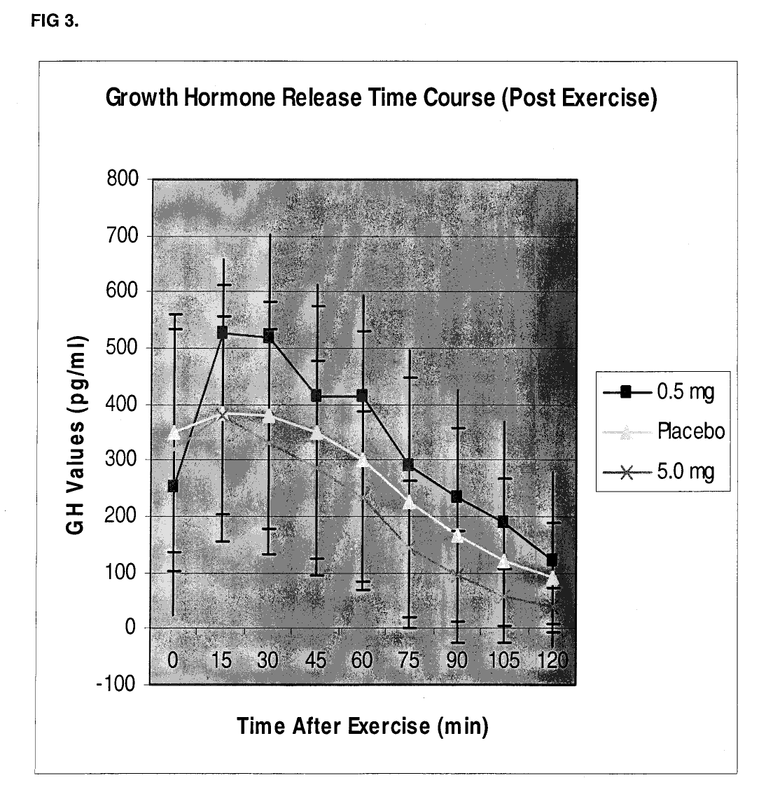

As illustrated in FIG. 1 a significant Group x Test interaction was observed (p=0.047) indicating the both melatonin supplementation and resistance exercise was effective in elevating the serum growth hormone levels. Pairwise comparisons showed that 5.0 mg melatonin supplementation, as per the experimental protocol, results in serum growth hormone levels being increased relative to placebo (p=0.017) during the pre-exercise period. There was no statistical difference between the 0.5 mg melatonin supplemented group and the placebo group (p=0.077) with respect to serum growth hormone levels during the pre-exercise period. Conversely, during the post-workout period both doses, 0.5 mg and 5.0 mg elicited statistically significant (p=0.045) elevations in serum growth hormone levels as compared to placebo. Surprisingly, the 0.5 mg melatonin supplemented group showed a greater increase in serum growth hormone levels as compared the group supplemented with the high dose of 5.0 mg of melatonin. FIG. 2, illustrates that the increase in growth hormone post-exercise was increased with statistical significance in the low dose group of 0.5 mg melatonin supplementation compared to placebo or the 5.0 mg dose groups (p<0.028). Furthermore, FIG. 3 shows a time course of serum growth hormone levels post-exercise with respect to the present invention. Although both groups showed an increase the serum levels of growth hormone from time T=15 minutes through to the completion of the experiment relative to placebo, the 0.5 mg melatonin supplemented group surprisingly showed an increased response to melatonin and exercise working synergistically to increase serum growth hormone levels greater than that of the 5.0 mg melatonin supplemented group.

Due to variability in baseline growth hormone values, the peak serum growth hormone responses both pre- and post-exercise were standardized to the participants respective baselines. As such, the relative change the serum growth hormone was used for statistical analysis. In accordance with what is known is in the art, the 5.0 mg of melatonin supplementation showed the greatest increase in serum growth hormone levels prior to exercise with an approximate increase of 2.35-fold. However, the 0.5 mg melatonin supplemented group showed no significant increase in serum growth hormone relative to placebo during the pre-exercise period. As can be seen in FIG. 1, the fold increase in serum growth hormone resulting from 5.0 mg melatonin supplementation is approximately the same in the post-exercise as it is during the pre-exercise period at 2.35-fold relative placebo. Interestingly, through, in the 0.5 mg Melatonin supplemented group, there is an approximately 3.16-fold increase in serum growth hormone levels relative to placebo during the post-exercise period. These data indicate that the lower dose of 0.5 mg of supplemented melatonin, but no the expected higher dose of 5.0 mg of supplemented melatonin works synergistically with exercise increase the serum levels of growth hormone. Doses of less that 5.0 mg of supplemented melatonin when administered prior to exercise show a greater increase in serum growth hormone levels than doses over 5.0 mg.

In the practice of the present invention melatonin also known as Melatonine, Circadin, N-Acetyl-5-methoxytryptamine, and 5-Methoxy-N-acetyltryptamine, may be administered to an individual in any pharmaceutically acceptable form or supplement as know in the art. The melatonin may be administered in the form of any pharmaceutically acceptable salt or ester. Furthermore, the melatonin may be administered in any pharmaceutically acceptable and somatically acceptable functional derivative thereof so as to fully practice the present invention as disclosed. Preferably, the melatonin is administered as melatonin.

The melatonin may be administered to an individual by any means known in the art. For example, the melatonin may be administered orally, intravenously, intramuscularly, interperitoneally, intracebroventricularly, transdermally or subcutaneously. The preferred method of administration as practiced by the present invention is oral administration.

The present invention also provides dosing in the range of about 0.01 mg to about 4.9 mg of melatonin or pharmaceutically acceptable derivative thereof wherein the active portion of the composition is within the dosing range. The preferred dose of the present invention is 0.5 mg of melatonin. Furthermore, the melatonin in the practice of the present invention said melatonin is administered to an individual by any of the aforementioned means and dosages prior to physical exercise. Preferably, the melatonin is administered by any of the aforementioned means and dosages approximately 60 minutes prior to exercise.

Although the following example illustrates the practice of the present invention in one of its embodiments, the example should not be construed as limiting the scope of the invention. Other embodiments will be apparent to one of ordinary skill in the art from consideration of the specifications.

Extensions and Alternatives

In the foregoing specification, the invention has been described with specific embodiment thereof, however, it will be evident that various modifications and changes may be made thereto without departing from the broader spirit and scope of the invention.

What is claimed:

1. A method for increasing growth hormone secretion in a mammal comprising the steps of:
the administration, to said mammal, of from about 0.01 mg to about 4.9 mg of melatonin or pharmaceutically acceptable derivative thereof;
supplemented with physical exercise.

2. The method claim 1, wherein said melatonin is in the form of a pharmaceutically acceptable salt.

3. The method claim 1, wherein said melatonin is in the form of a pharmaceutically acceptable ester.

4. The method of claim 1 wherein said melatonin is administered to said mammal orally.

5. The method of claim 1 wherein said melatonin is administered to said mammal intravenously.

6. The method of claim 1 wherein said melatonin is administered to said mammal intramuscularly.

7. The method of claim 1 wherein said melatonin is administered to said mammal interperitoneally.

8. The method of claim 1 wherein said melatonin is administered to said mammal intracerbroventricularly.

9. The method of claim 1 wherein said melatonin is administered to said mammal transdermally.

10. The method of claim 1 wherein said melatonin is administered to said mammal subcutaneously.

11. The method of claim 1 wherein said melatonin is administered to said mammal rectally.

12. The method of claim, wherein said melatonin is administered to said mammal prior to said exercise.

13. The method of claim 1, wherein said melatonin is administered to said mammal between commencement of said exercise and up to about 60 minutes prior to commencement of said exercise.

14. The method of claim 1, wherein said exercise is undertaken at a level of at least 50% of the maximum output of said mammal.

15. The method of claim 1, wherein said exercise is undertaken at a level of at least 80% of the maximum output of said mammal.

16. The method of claim 1, wherein said exercise is undertaken at a level of at least 85% of the maximum output of said mammal.

17. The method of claim 1, wherein between 0.01 to 4.00 mg of said melatonin is administered to said mammal.

18. The method of claim 1, where between 0.01 to 3.00 mg of said melatonin is administered to said mammal.

19. The method of claim 1, where between 0.01 to 2.00 mg of said melatonin is administered to said mammal.

20. The method of claim 1, where between 0.01 to 1.00 mg of said melatonin is administered to said mammal.

21. The method of claim 1, where between 0.01 to 0.50 mg of said melatonin is administered to said mammal.

22. The method of claim 1, where about 0.50 mg of said melatonin is administered to said mammal.

23. A method for increasing growth hormone secretion in a mammal comprising the steps of:
administration of about 0.50 mg of melatonin or pharmaceutically acceptable derivative thereof up to 60 minutes prior to physical exercise; and
performing physical exercise at a level of at least 85% of the maximum output of said mammal.
